(19) United States
(12) Patent Application Publication
(10) Pub. No.: US 2013/0065866 A1
(43) Pub. Date: Mar. 14, 2013

(54) DENTINAL DRUG DELIVERY COMPOSITION AND SCREENING METHOD

(71) Applicant: University of Medicine and Dentistry of New Jersey, Somerset, NJ (US)

(72) Inventors: Kenneth Markowitz, Fanwood, NJ (US); Evros Vassiliou, Franklin Lakes, NJ (US)

(73) Assignee: UNIVERSITY OF MEDICINE AND DENTISTRY OF NEW JERSEY, Somerset, NJ (US)

(21) Appl. No.: 13/670,873

(22) Filed: Nov. 7, 2012

Related U.S. Application Data
(63) Continuation-in-part of application No. 13/511,008, filed on May 29, 2012.
(60) Provisional application No. 61/263,510, filed on Nov. 23, 2009.

(51) Int. Cl. A61K 31/202; A61K 31/192; A61K 47/04; A61K 47/46; A61K 31/155; A61K 31/5383; C07C 69/157; C07C 57/30; C07C 57/03; C12Q 1/02; C12Q 1/68; G01N 33/566; A61P 29/00; A61P 31/00; A61P 1/02; A61K 31/616

(52) U.S. Cl. 514/165; 514/570; 514/560; 514/770; 514/782; 514/635; 514/230.2; 560/143; 562/496; 554/224; 435/29; 435/6.13; 435/6.11; 435/6.12; 435/7.92; 435/7.1

(57) ABSTRACT

The present invention is a dentinal drug delivery composition composed of cationic and/or neutral porous particles containing an effective amount of a therapeutic agent, a method for using the dentinal drug delivery to provide a dental treatment, and a method for identifying anti-inflammatory agents capable of diffusing through dentin.

### INTRODUCTION

This application is a continuation-in-part application of U.S. patent application Ser. No. 13/511,008, filed May 29, 2012, which is a U.S. National Stage Application of PCT/US2010/057718 filed Nov. 23, 2010 and claims benefit of priority to U.S. Provisional Application Ser. No. 61/263,510 filed Nov. 23, 2009, the contents of each of which are incorporated herein by reference in their entirety.

### BACKGROUND OF THE INVENTION

Dentin is a biological composite composed of both inorganic and organic components. The chemical and physical characteristics of surgically exposed dentin determine the ability of therapeutic and restorative materials to adhere to these surfaces. In vital-permeable dentin there is a continuous outward-flow of dentin fluid. Materials applied to the dentin surface may be able to penetrate the dentinal tubules but fail to be retained if they are incompatible with this fluid or are dislodged by its flow. When dentin surfaces are demineralized by acid etching, a layer of collagen fibers are exposed. Hydrophilic dentin bonding agents can interpenetrate this hydrated collagen layer resulting in a strong bond between dentin and restorative materials (Nakabayashi (1992) Proc. Finn. Dent. Soc. 88 Suppl 1:321-9). Hydrogen bonding and van der Waals forces have been found to be responsible for the interaction between collagen and several types of dentin primers. Hydroxyethylmethacrylate (HEMA) can associate with collagen through sites on the collagen molecule that act as ligands for this polymer (Vaidyanathan, et al. (2003) J. Adhes. Dent. 5:7-17). In addition, agents such as gluteraldyhyde can covalently link primers to the dentin collagen.

Electrostatic interactions may help anchor restorative and other therapeutic materials to dentin. Many biological surfaces have fixed negative charges due to the presence of proteins, and other macromolecules, containing carboxylated, sulfonated or phosphorylated functional groups that are ionized at physiological pH. For example, clean hair has a net anionic surface charge (Ungewiss, et al. (2005) Anal. Bioanal. Chem. 381:1401-7). Several types of cationic polymers containing quaternary nitrogen functional groups adhere to hair and are used as conditioners. Cationic antimicrobial agents such as chlorhexidine are substantive to many oral surfaces including dentin (Rosenthal, et al. (2004) Oral Surg. Oral Med. Oral Pathol. Oral Radiol. Endod. 98:488-92). As is the case with other biological substrates, utilization of electrostatic forces should help attach material to dentin surfaces. The mineral phase of dentin provides other means by which organic molecules can bind to this tissue. Organic acids can both demineralize and bind to dentin. Many low molecular weight organic acids such as citric and lactic acid form soluble calcium salts. These acids demineralize dentin (Yoshida, et al. (2001) J. Dent. Res. 80:1565-9). Higher molecular weight polyalkenoic acids bind exposed calcium ions on the dentin surface forming tenacious gels that resist removal. Calcium ions in the fluid layer adjacent to the dentin surface can also contribute to adhesion by forming ionic cross-bridges between polymers containing organic acid groups and anionic macromolecules in dentin (Hannig & Hannig (2009) Clin. Oral Investig. 13:123-39). Since dentin possesses a variety of chemical functionalities, different binding mechanisms can be employed to attach materials to the dentin surface.

Dentin has a markedly inhomogeneous structure. The dentinal tubules become wider and are more numerous in deep dentin, close to the dental pulp. Since each tubule has a thin sheath of organic material (the lamina limitans) lining the lumen of each tubule, the density of organic material would be expected to be greater in deep as opposed to shallow dentin (Thomas (1984) J. Dent. Res. 63:1064-6). In addition to collagen, the dentin contains a variety of other proteins such as dentin phosphosphoryn, which are highly phosphorylated and have low pKa values (Butler (1995) Connect. Tissue Res. 33:59-65). These proteins are believed to play a role in dentin mineralization and when decomplexed from calcium by etching would become anionic. In addition proteoglycans are present particularly in the predentin (Waddington, et al. (2003) Matrix Biol. 22:153-61).

The chemical characteristics of dentin's intratubular organic material can affect the diffusion of solutes through dentin. Most low molecular weight anionic materials such as pertechnetate and iodine ions diffuse readily through dentin (Pashley, et al. (1977) J. Dent. Res. 56:83-8). In contrast, chlorhexidine, a cationic agent, has a lower dentin permeability coefficient than is predicted on the basis of its molecular weight (Pashley & Livingston (1978) Arch. Oral Biol. 23:391-5). A basic polypeptide (parathyroid hormone; Pichette, et al. (2000) J. Chromatogr. A 890:127-33) was found to be unable to diffuse through dentin unless it was absorbed onto albumen, an anionic protein (Pashley (1988) Int. Endod. J. 21:143-54). The results of these diffusion experiments indicate that dentin binds cationic molecules hindering their transdentinal diffusion. This behavior is consistent with the view that the etched-dentin surface and tubule walls have fixed anionic charges and provide binding sites for cationic materials.

Alternatively, it has been proposed that the dentinal tubules are filled with a cationic gel of unspecified composition (Linden, et al. (1995) Arch. Oral Biol. 40:991-1004). This gel was observed using scanning-probe microscopy. In in vitro experiments measuring dentin flow, treatment of dentin slices with proteolytic enzymes resulted in a large increase in flow, indicating that proteins including collagen partially reduced the effective diameter of the dentinal tubules (Linden, et al. (1995) supra). Transdentinal diffusion of negatively charged myoglobin was restricted as compared to neutral myoglobin even though both proteins had the same ionic radius. These observations lead these investigators to conclude that the intertubular gel had fixed positive charges.

Using the scanning electron microscope (SEM), solid polystyrene beads with cationic surface charges were observed to adhere to cut etched dentin surfaces and were observed in some dentinal tubules (U.S. Pat. No. 5,211,939).

### SUMMARY OF THE INVENTION

The present invention is a dentinal drug delivery composition composed of cationic and/or neutral porous particles containing an effective amount of a therapeutic agent, wherein the particles are in admixture with a carrier suitable for attachment of the particles to the dentin. In one embodiment, the porous particle is composed of silicon or latex. In other embodiments, the therapeutic agent is an anti-inflammatory drug that controls pulpal inflammation; an antibiotic; or an analgesic. A method for providing dental treatment with the composition of this invention is also provided.

This invention also provides a method for identifying an anti-inflammatory agent for decreasing inflammation in dental pulp. This method includes the steps of contacting a coronal surface of an isolated dentin sample with a test agent, incubating the test agent and dentin sample so that diffusate is formed, contacting an isolated dental pulp cell with the diffusate, and determining whether the diffusate has a sufficient amount of test agent to decrease the expression or activity of at least one inflammatory mediator of the dental pulp cell thereby identifying an anti-inflammatory agent for decreasing inflammation in dental pulp. In some embodiments, the test agent is a non-steroidal anti-inflammatory drug or an ω-3 fatty acid.

### BRIEF DESCRIPTION OF THE DRAWINGS

FIG. 1 shows the percent open tubule (6 observations each group) for control (untreated) COOH, NH₂ and OH treated dentin using 2% silica beads.

FIG. 2 depicts the use of the instant drug delivery composition to reduce pain, inflammation and infection in teeth being treated for deep decay.

FIG. 3 depicts the application of the instant drug delivery composition for delivering therapeutic agents in dental restoration.

### DETAILED DESCRIPTION OF THE INVENTION

Within each vital tooth there is a small organ rich in nerves and blood vesicles called the dental pulp. Disease such as caries or trauma can cause inflammation in this delicate tissue. Pulpal inflammation often results in the severe pain commonly referred to as toothache. Since the pulp is enclosed within the dental hard tissues, the circulatory events occurring during severe inflammatory processes frequently result in loss of blood supply and tissue death. Even mild inflammatory responses such as those occurring following the placement of a routine dental restoration can result in post-operative sensitivity. Conventional treatment of pulp inflammation is limited. Patients with severe pulp inflammation require removal of the offending tooth or removal of the pulp (endodontic therapy, also known as root canal treatment) with conservation of the tooth. Endodontic therapy is expensive and time consuming; hence by increasing the ability to manage pulpal inflammation pharmacologically, dental care is improved.

It has now been found that porous particles are of use in delivering, e.g., anti-infective or anti-inflammatory drugs as well as biological agents such as growth and biological response modifying factors to diseased and injured dental pulps in order to improve treatment outcomes and allow dental procedures to be delivered in a more predictable and cost effective manner. Once attached to the dentin, these particles can release drug that reaches the site of action by diffusing through the fluid-filled tubules that are a prominent component of the dentin structure. The drug carrying particles can be nondegradable and form part of the interface between the dentin and the restorative material. Procedures for fabricating porous silica particles over a range of clinically useful particle diameters have been described in the literature and the materials are commercially available.

Accordingly, the present invention features a dentinal drug delivery composition composed of cationic or neutrally charged porous microparticles containing an effective amount of a therapeutic agent, wherein said particles are in admixture with a carrier suitable for attachment of the microparticles to the dentin. As used herein, the terms “microspheres,” “particles” and “microparticles” are essentially synonymous. These microspheres, which are generally spherical in shape, are typically sized to have nominal diameters in the range of from about 0.1 micron to about 1 micron. In particular embodiments, the average diameter of the microspheres of the invention are about 0.5 micron.

By “porous” is generally meant a porosity of at least about 50%, and preferably a porosity of from about 50% to about 65%. The degree of porosity refers to the total pore volume within the solid support, e.g., silica particle. Porosity increases with increasing pore volume.

The porous microspheres of the present invention can be prepared by any conventional method. In particular embodiments, the microsphere of the invention is composed of silica or latex. By way of illustration, the silica particles can be prepared by spray drying silica solutions made by the controlled hydrolysis of tetraethyl-o-silicate or similar organic silicon compounds. This method allows the formation of highly purified porous silica microspheres at a relatively low cost and with highly controlled properties. The silica support can be made with different particle sizes and different pore sizes to, e.g., accommodate the therapeutic agent being delivered.

More specifically, appropriate silica sols can be prepared by the hydrolysis of organic silicates in the manner described by Stöber, et al. (1968) J. Colloid and Interface Science 26:62-69. This approach is known to make silica sols with a very high purity and with a narrow sol particle size distribution. The particle size of the sol prepared in this manner determines the pore size of the porous silica microspheres ultimately made from these sols, with the average pore size being about one-half the average diameter of the silica sol microparticles.

Porous silica microspheres can then be made from these aqueous colloidal silica sols by using well known spray-drying equipment and methods (Masters, Spray Drying Handbook, 5^{th} ed. Longman Scientific and Technical, New York (1991)). In some embodiments, the silica solutions are first be flocculated or partially pre-gelled by using a process such as described in Iler, The Chemistry of Silica, Chapter 4, John Wiley, New York (1979), to produce microspheres with a porosity that is higher than that available by the direct spray drying of silica sols. The concentration of the silica solution, the type and rate of the spray-drying nebulization (for example, two-fluid nozzle or spinning disk), the drying temperature, the rate of heated air supply, and the like, are all adjusted to produce the porous silica microspheres of the desired size and size distribution.

In particular embodiments, porous silica particles contain functional groups on the surface thereof that impart a neutral or positive charge. A cationic charge on the surface of the instant particles can be incorporated by conventional methods using positively charged groups selected from the group of primary amine, secondary amine, tertiary amine, quaternary ammonium salts, amidines, pyridinium salts and mixtures thereof. Exemplary groups include amine and amidine groups.

Latex particles with engrafted cationic surfaces are also embraced by the present invention. Suitable latex particles may be obtained commercially from Interfacial Dynamics Corp., Portland, Oreg.

The particle sizing may be accomplished by a number of well-known methods, such as sieving, air classification, and liquid elutriation. Sieving is the simplest and least costly method. However, this method produces products that have the greatest concentration of fine particles, because of the tendency of fines to adhere to larger particles and therefore not be properly fractionated. Air classification with a relatively expensive machine is a convenient method that permits a high throughput of desired particles to be fractioned accurately in a narrow particle size distribution.

It is further contemplated that the microspheres of the invention can be coated with various inorganic and organic constituents in order to impart the particles with affinity for surgically prepared and pathologically altered dentin and/or the ability to hold and release various pharmacological agents.

In contrast to the absorption of a therapeutic substance on the surface, the instant microspheres are porous and find application in holding and releasing a variety of therapeutic agents used in the field of dentistry. Examples of topically active drugs that could be released from a microsphere of the invention include, but are not limited to, analgesic agents; anti-inflammatory agents such as 2-amino-3-benzoylbenzeneacetamide (NEPAFENAC), acetylsalicyclic acid (aspirin), ibuprofen, diclofenac, difusinol, indomethacin, an ω-3 fatty acid or other monounsaturated or polyunsaturated fatty acid (e.g., oleic acid or docosahexaenoic (DHA)); anti-microbial agents such as chlorhexidine; and fluoroquinolone antibiotics such as ofloxacin.

For use in vivo, particles of the present invention can be prepared as pharmaceutical compositions, wherein the particles are in admixture with a carrier suitable for attachment of the particles to the dentin. In this respect, the carrier selected does not change or mask the charge of the particle. Suitable carriers include, but are not limited to water, saline or other conventional carrier used in dental applications.

The ability of the instant porous microspheres to adhere to dentin and release drugs can be examined via a variety of in vitro methods. For example, the ability of a porous particle to adhere to dentin surfaces can be assessed using an in vitro assay as described herein. Part of the intertubular dentin matrix and the walls of the tubules are composed of organic macromolecules. In this respect, adherence of dyes and particulate material to dentin can be readily assessed by photomicrographic means. As shown here, untreated dentin stained with toluidine blue has a deep purple-blue color that is indistinguishable from the dye solution itself. The pattern of dentin's staining with toluidine blue has been described in demineralized, dehydrated tissue (Major (1966) Arch. Oral Biol. 11:1293-305). The intratubular dentin very close to the pulp stains intensely. Moving away from the pulp, a poorly stained region is encountered, then a zone where material inside the tubule lumen is stained. The superficial dentin is observed to be lightly stained. The biochemical nature of the tissue determines the color resulting from toluidine blue staining. Large amounts of acidic molecules in tissue effect the orientation and spacing of dye molecules resulting in a color shift to red. This phenomenon is called metachromasia and was observed in histological sections of developing mouse dentin (Ravindranath & Basilrose (2005) Acta Histochem. 107:43-56). Treatment of the tissue sections with enzymes that cleave acidic functional groups off of proteins reduces the degree to which metachromasia is observed, indicating that this type of staining is very sensitive to the chemical composition of tissue.

The heavy orthochromatic staining observed herein with toluidine blue indicates that dentin is rich in anionic molecules. In the dentin sections cut perpendicular to the long axis of the tooth, dentin overlying the pulp horn area was observed to be particularly deeply stained. This is the region of dentin with the widest tubules, the highest permeability (Pashley, et al. (1987) Arch. Oral Biol. 32:519-23) and the greatest density of intratubular organic material. In vivo, the outward flow of dentin fluid limits the diffusion driven penetration of dyes through the tubules. The experiments in this study were performed without the application of simulated pulp pressure, since the goal was to use dye staining as a means to examine the binding properties of dentin. Since extracted teeth were used in this study, organic material from the dental pulp and remnants of the odontoblasts may have leaked into the tubules and increased the intensity of the staining. The enamel portions of the tooth slices stained lightly with toluidine blue indicating that the dye has some affinity for hydroxyapatite. Similar light staining can also be observed when squares made of sintered hydroxyapatite are treated with toluidine blue.

In marked contrast to untreated dentin, dentin that was pretreated with the cationic polymer solution was only faintly stained. Chroma meter readings indicated that unstained dentin has a high brightness value and a positive reading on the yellow-blue scale indicating yellow color. Following toluidine blue staining, the brightness value significantly dropped and a significant shift in the yellow blue parameter occurred to a negative value, indicating that the dentin color had become blue. Brightness values from dentin surfaces treated first with the cationic polymer than stained with toluidine blue were not significantly different from those of unstained dentin. The yellow-blue scale reading of dentin that was polymer treated then toluidine blue stained was significantly lower than that of unstained dentin, indicating that some shift in dentin color from yellow to blue occurred. This pale blue staining with more intense staining of the pulp horn region was evident in the cationic polymer-treated stained dentin. These results showed that treatment of the dentin with the cationic polymer reduced, but did not entirely block toluidine blue staining.

The ability of porous particles, found to have affinity for dentin in the experiments described above, can be examined in conventional release assays as described in the literature. Moreover, the transdentinal diffusion of drug released from particles can be measured. Due to the chemical properties of the dentin, the transdentinal diffusion of certain solutes is restricted. Dentin sections can be placed into a dentin diffusion cell. Loaded particles can be applied to the outer dentin surface while the fluid on the inner (pulpal) side of the disk is withdrawn at regular intervals and analyzed for the presence of the drug. This experiment allows for the evaluation of drug release and delivery kinetics.

The findings herein have significant clinical applications. Therefore, the instant invention also includes a method for providing dental treatment by administering to a subject in need thereof, the dentinal drug delivery composition of the invention. The observation that charged acidic particulates and cationic materials adhere to etched dentin indicates that these types of agents can be incorporated into restorative materials or agents used to topically desensitize teeth. Charged particulates can also be used as dentin drug delivery compositions, where drugs can be loaded into hollow or porous microspheres that can release the drug into the dentinal fluid in a diffusion controlled manner over a period of time. Following trauma or the excavation of deep decay, pulpal inflammation can cause pain and eventual loss of tooth vitality. New insights into the peripheral mechanisms of dental pain can lead to the development of new analgesic drugs that target peripheral intradental nerve endings. When dentin (particularly deep dentin) is etched, fluid flows in an outward direction through the patent tubules. This outward flow opposes the inward diffusion of solutes, including drugs, through the dentin. Following placement of a bonded restoration over the exposed dentin the outward flow ceases. Placing the instant drug delivery composition between the dentin and the restorative material would allow the drug to diffuse into the dentinal fluid without the opposing influence of this outward fluid flow. Drug delivery compositions that have an affinity for deep dentin can be applied as part of the restorative treatment releasing drug into the dentinal fluid while serving as the interface between the dentin and the bulk of the restoration (FIG. 2).

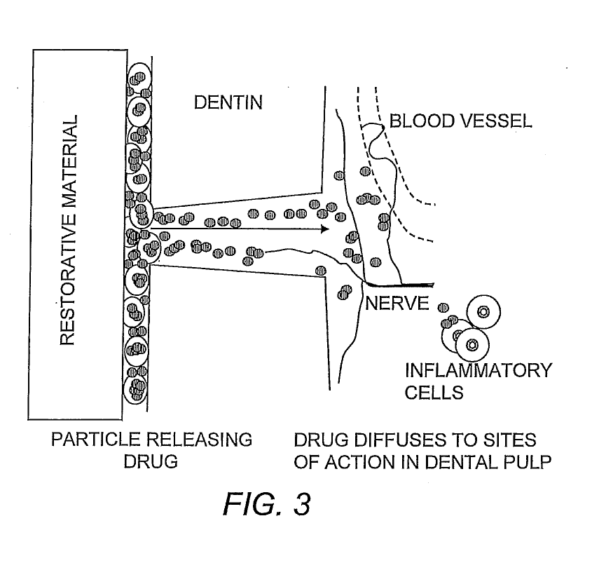

The use of hollow or porous silica particles as drug carriers is illustrated in FIG. 3. Following the removal of deep tooth decay, the dentist would place the particulate containing drug delivery composition onto the exposed dentin. The drug carrier would then be covered by the tooth restorative material. Once in contact with the dentin, drug would diffuse out of the particle and through the dentinal fluid to its site of action in the dental pulp. The tooth dentin is composed of a partially mineralized matrix containing collagen and other proteins. Fluid filled tubules, 0.5-2.5 μm in diameter transverse the thickness of the dentin. The instant delivery composition can be designed so that it adheres to the dentin and releases the pharmacological agent, which subsequently diffuses through the fluid filled tubules to the neural, vascular or other sites of action in the superficial pulp tissue. Since the acute phase of pulp inflammation peaks about one week after injury, drug delivery for that period of time would be effective in modulating the inflammatory response. Based on in vitro experiments utilizing 0.5 μm latex spheres, as described herein, the surface characteristics of particulate materials that adhere to surgically prepared dentin surfaces have now been defined. Due to the heterogeneous nature of the dentin, beads with a variety of surface chemistries were found to have affinity for this tissue.

For drugs to be effective, they must be capable of diffusing through the dentinal tubules. Dentin acts a permselective membrane where electrostatics, aqueous solubility and molecular weight mediate diffusion of a molecule through the dentin. Therefore, to identify agents that can diffuse through a dentin barrier in sufficient amounts and act on dental pulp cells, this invention also provides an in vitro cell-based method for assessing drug diffusion through dentin and activity against dental pulp cells. Determining whether agents can diffuse through dentin is critical in determining which agents can be useful in a dentin drug-delivery system and provide therapeutics to the dental pulp following invasive dental processes.

Agents that can be screened using this method of the invention include, but are not limited to, analgesic agents; anti-inflammatory agents such as 2-amino-3-benzoylbenzeneacetamide (NEPAFENAC), acetylsalicyclic acid (aspirin), ibuprofen, diclofenac, difusinol, indomethacin, an ω-3 fatty acid or other monounsaturated or polyunsaturated fatty acid (e.g., oleic acid or docosahexaenoic (DHA)); anti-microbial agents; and antibiotics. Moreover, combinations of drugs and dosage forms can be evaluated using the method of this invention. Synergies between anti-inflammatory and water-soluble analogs of ω-3 fatty acids can also be examined.

This screening method of the invention includes the steps of contacting a coronal surface of an isolated dentin sample with a test agent, incubating the test agent and dentin sample so that diffusate is formed on the pulpal side of the dentin sample, contacting an isolated dental pulp cell with the diffusate, and determining whether the diffusate contains a sufficient amount of test agent to act on a cell of the dental pulp. In one embodiment, it is determined whether the diffusate contains a sufficient amount of test agent to inhibit or decrease the expression or activity of at least one inflammatory mediator of the dental pulp cell (e.g., nociceptive nerve endings, blood vesicle cells or inflammatory cells).

The dentins sample can be isolated by conventional methods and used in combination with a commercially available two-chamber diffusion cell, wherein the partition separating the two chambers holds the dentin sample (e.g., a dentin disk) in an orientation so that the dentin tubules provide the only connection between the two chambers. See, e.g., Hanks, et al. (1993) J. Dent. Res. 72:931-938; and Puapichartdumrong, et al. (2003) Internatl. Endodont. J. 36:674-81. The diffusant (i.e., test agent) is loaded into one cell (the donor compartment) then following one or more time intervals, the presence and concentration of the diffusant is ascertained in the second cell (receptor compartment). Test agent found in the receptor compartment during the course of the experiment reached that chamber by diffusing through the dentin barrier.

After the one or more time intervals (e.g., one or more minutes, hours, or days), the diffusate is collected and tested for activity against an isolated dental pulp cell or isolated population of dental pulp cells. An isolated pulpal cell or population of dental pulp cells refers to cells that are removed from their native environment (i.e., the dental pulp). The isolated cells can be cultured primary cells or cultured cell line and include, but are not limited to fibroblasts, odontoblasts, histiocytes, macrophage, granulocytes, mast cells or plasma cells. Exemplary cell lines include, but are not limited to the murine macrophage cell line, RAW 264.7; immortalized mouse odontoblast cell line, MDPC-23; and the murine fibroblast cell line, 3T3.

As indicated, in some embodiments it is determined whether the diffusate contains a sufficient amount of test agent to inhibit or decrease the expression or activity of at least one inflammatory mediator of the dental pulp cell. In accordance with this embodiment, the isolated pulpal cell or population of dental pulp cells can be challenged with an inflammation-provoking stimulus (e.g., Lipopolysaccharide (LPS), N-formyl-methionyl-leucyl-phenylalanine, or phorbol myristate acetate) either before or after being contacted with the diffusate, so that the effect of the drug diffusing through the dentin barrier on inflammatory mediator production is assessed.

Prostaglandins such as PGE2 and nitric oxide (NO) are inflammatory mediators that are generated in injured tissue and influence vascular tissues, nociceptive nerve endings and immune cells. For example, exposure of immune and other cells to bacterial toxins such as LPS evokes production of PGE2 and NO. Other known mediators of inflammation include, but are not limited to, bradykinin, histamine, leukotrienes (B_{d}, C_{d}, D_{d}, E_{d}) IL-8, and neutrophil and macrophage lysosomal enzymes. The effect of test agents on the expression or activity of one or more of the above-referenced inflammatory mediators can be determined by conventional methods. For example, mRNA expression can be determined by dot blot, northern blot, or reverse transcriptase (RT)-PCR; protein expression can be determined by western blot or ELISA analysis; and NO production can be determined using a Griess reaction assay (e.g., EMSNOTOT kit) or ozone-based chemiluminescence assay.

A reduction or decrease in the level or activity of one or more inflammatory mediators as compared to a control cell or population of cells, e.g., cells not contacted with the test agent, indicates that a sufficient amount of the test agent diffused through the dentin to act on the isolated cell or population of cells of the dental pulp. By measuring the responses of cells to a series of known drug doses, a dose response curve can be generated. The dose response curve can be used to calculate the drug concentration generated in the second cell (receptor compartment) in individual diffusion experiments. Data collected in these experiments can be used to determine the transdentinal flux of individual drugs using Fick's equation:

Flux=−P·A·(cB−cA)

where: A is the area of the dentin disk available for diffusion; (cB−cA) is the difference in concentration between the second (cB) and first (cA) chambers, and P is the dentin permeability of the drug. A high concentration of drug is added to the first chamber and media in the second chamber can be periodically replaced with drug-free media so that the term (cB−cA) will approximate—cA.

Anatomic factors influence P since P∝1/L, where L is tubule length. This can be standardized using dentin slices of the same thickness. The diameter of the tubule is an important determinant of flux since, P∝r2, the tubule radius raised to the second power. By using teeth from young subjects, the value of this term can be maximized since the tubules narrow as people age. By controlling for these anatomic factors it is possible to define the conductance of the dentin to the particular drug. Electrostatic charge and solubility influence conductance. Since the tubule diameter is quite large compared to molecular dimensions, molecular weight has little effect on transdentinal diffusion over a range of sizes including those of most anti-inflammatory drugs. The large molecular size and other characteristics of lipids-based agents may have a limiting effect on their transdentinal flux.

Using the method of this invention, agents that exhibit potent inhibition of inflammatory responses and cross the dentin barrier (i.e., a high P value) can be identified. Agents that efficiently diffuse through the dentin can be used in combination with the delivery system described herein to provide patients with more comfortable, cost effective and reliable dental care, e.g., following invasive dental processes or dental caries.

The invention is described in greater detail by the following non-limiting examples.

### Example 1

### Materials and Methods

Dentin Specimen Preparation. Caries-free human third molars from subjects less than 30 years of age were used in this study. Teeth were collected in 1% phenol then debrided of adherent hard and soft tissue and externally sterilized by soaking in chlorine bleach for 2 minutes followed by 2% hydrogen peroxide for 2 minutes. Since the pulp space was not sterilized, the teeth were handled with infection control precautions throughout the experimental procedure. Teeth were used within one month of collection.

The teeth were sectioned perpendicular to their long axis, using a low speed saw (ISOMET, Buehler Ltd., Lake Bluff, Ill.) with a diamond blade and deionized water lubricant. One-half millimeter thick sections, free of occlusal enamel, were obtained. Since there was particular interest in deep dentin in this study, some sections were partially perforated by pulp horns. A grove was made on the pulpal side of each disk using an abrasive disk on a low speed dental handpiece (MTI Precision Products, Lakewood, N.J.) in order to facilitate later fracturing. For disks that were examined with the SEM, the occlusal side was polished with a series of abrasives to remove striations left behind by the diamond blade (all polishing supplies from Buehler). Initially, 600 grit silicon carbide paper was used with water lubrication, followed by an aqueous slurry of 3 μm diamond paste on a felt pad and finally an aqueous slurry of 0.25 μm diamond paste on a felt pad. The polished disks were sonicated for 2 minutes (model 450 SONIFIER, Branson, Danbury, Conn.) at 30% power setting and 50% duty cycle. The disks were then etched for 2 minutes in 0.5 M EDTA at pH 7.4 with agitation. The disks were rinsed in 0.9% NaCl and stored in 0.9% NaCl until use. Immediately prior to the application of dye or other experimental agents, the disks were fractured in half along the grove prepared on the pulpal side of the section. All experimental treatments were applied to the occlusal surface of the dentin specimen.

Dye Staining.

Nine tooth slices were etched with EDTA and fractured in half as described above. Immediately prior to use, the disks were rinsed in deionized water. The color of the untreated dentin specimens were examined with a tristimulus color analyzer (model CR221 Chroma meter, Minolta, Osaka, Japan). This device measures three dimensions of a specimens color: the brightness, as well as two aspects of chromaticity, position on an axis representing red-green and position on an axis representing yellow-blue. Since in these experiments light-yellow dentin was stained with a dark-blue dye, the brightness values and position on the yellow (positive numbers)-blue (negative number) axis were judged to be the relevant color parameters and recorded for analysis. These chroma meter readings were taken from three positions on each dentin surface using a 3 mm diameter-measuring tip and specimen holder. One half of each disk was then soaked for 60 seconds in a 33 weight % of the cationic polymer polyquaternium-6 in 67 weight % of water, while the other half was soaked in deionized water. Both disk halves were then rinsed with a gentle stream of deionized water for 20 seconds, then placed into separate vials containing a 0.5% solution of toluidine blue-0 (molecular weight=305.83) (Harleco, Philadelphia, Pa.) for 1 minute. The polymer and water-treated disk halves were then rinsed with deionized water for 1 minute, blotted dry and reexamined, under identical light conditions, with the color analyzer with three readings taken from each disk half.

Three other dentin specimens were prepared and treated as described above. Following toluidine blue staining, the two halves of each dentin disk were photographed (DP12 Microscope Digital Camera System, Olympus, Tokyo, Japan) side by side (occlusal surface up) in order to visually compare the intensity of staining between the polymer and water treated halves of the tooth slice.

Latex Beads and Scanning Electron Microscopy (SEN).

Polished, EDTA-etched dentin disks were fractured in half and rinsed in deionized water prior to use. As with the dye staining experiments, one half of each disk was soaked in the cationic polymer solution for one minute while the other half was exposed to deionized water. Both disk halves were rinsed in deionized water for 20 seconds. The two disk halves were then soaked in a 4 weight % percent dispersion of either anionic or cationic latex beads in water for one minute in separate bottles. The disk halves were then gently rinsed with deionized water for 20 seconds and allowed to air-dry for at least 24 hours prior to preparation for SEM analysis. The two dentin disk halves from each disk (one polymer treated before bead application the other water treated) were attached to aluminum SEM sample mounts with silver paint (Ted Pella Industries, Reading, Calif.), then sputter coated with gold using an SEM Coating System (BIO-RAD, Hercules, Calif.), and examined using a S-2500 Scanning Electron Microscope (Hitachi, Pleasanton, Calif.). The split dentin disks were examined under low magnification (35× magnification) and areas that were near the center of the disk and symmetrically situated on either side of the fracture were selected for higher magnification examination and photomicrography. In this way, the effects of cationic polymer application on bead adhesion could be examined in areas of equivalent tubule morphology (Ahmed, et al. (2005) J. Oral Rehabil. 32:589-97). This procedure was conducted on 11 tooth slice pairs, five for the cationic and six for the anionic beads, respectively. The number of beads in a representative high power (6000 magnification) image from each of 22 disk halves examined was counted by an investigator who did not know what treatment was applied, with the aid of the particle counter tool in the public domain program, NIH Image. In this study the enamel edges of the tooth slices were used to handle the specimens, hence the ability of the beads to adhere to enamel was not examined.

Experimental Treatments.

The cationic polymer used in this study was a 33 weight % water solution of (poly) 2-propen-1-aminium, N,N-dimethyl-N²-propenyl chloride, CAS No. 26062-79-3 (MERQUAT 106 Nalco Company, Naperville, Ill.). The polymer is composed of repeating cationic quaternary nitrogen groups with the following unit structure.

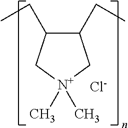

This polymer was selected for these experiments because of its high cationic charge density. This type of polymer has affinity for hair and is used in commercial products under the designation polyquaternium-6. The 33 weight % in 67 weight % water solution used in these experiments had a low viscosity and was readily washed off the dentin surface.

The two types of latex polymer beads used in these experiments were manufactured by Interfacial Dynamics (Eugene, Oreg.). Both had a particle diameter of approximately 0.5 μm. Cationic beads had amidine surface groups and anionic beads had carboxylic acid groups. The anionic and cationic beads used had equivalent charge densities on their surfaces. Both the amidine and carboxylate beads were used as 4 weight % surfactant-free aqueous dispersions, in which the charge on the individual particles stabilized the emulsion.

Data Analysis.

Chroma meter readings and bead counts were determined as mean±standard deviation. A one way analysis of variance (ANOVA) with a pair-wise Tukey-Kramer test was performed using the JMP statistical program (SAS Institute Inc., Cary, N.C.) in order to determine if significant differences existed in brightness values and yellow-blue values between unstained dentin, dentin that was treated with polymer prior to toluidine blue staining and stained dentin that was not polymer treated. The same statistical test was used to determine if there was a significant difference in bead counts per high power field between water-treated dentin exposed to the cationic beads, polymer-treated dentin exposed to the cationic beads, water-treated dentin exposed to the anionic beads, and polymer-treated dentin exposed to the anionic beads. Significance was set at the p<0.05 level.

### Example 2

### Dye Staining

Prior to staining, the right half of a tooth slice was treated with the cationic polymer solution and the left half was treated with deionized water. The water-treated part of the slice showed intense blue staining of the dentin and light enamel staining. The stained dentin was the same color as the dye solution indicating orthochromatic toluidine blue staining. In initial studies, it was observed that dentin from deep sections stained more intensely than shallow dentin and the dentin that was occlusal to the pulp horns stained more intensely than dentin under the occlusal fissures. On the half of the slice treated with the cationic polymer, faint blue staining could be seen particularly close to the pulp horns. Overall, the cationic polymer-treated enamel was largely unstained. In two other sets of split dentin disks, the halves treated with cationic polymer were stained much less intensely than the water-treated halves.

Chroma meter readings obtained from dentin prior to stain application and from stained dentin with and without polymer application were obtained. Unstained dentin had a high chroma meter lightness value of 86.4±4.7. Water-treated dentin had a significantly (p<0.05) reduced lightness value of 20.1±5.5 when stained with toluidine blue. In contrast, dentin that was treated with the cationic polymer prior to dye staining had a brightness value of 83.2±5.5; however, this value was not significantly (p>0.05) different from the value for unstained dentin, but was significantly (p<0.05) higher than the value for dentin that did not receive the cationic polymer treatment prior to staining. Unstained dentin had a chroma value on the yellow-blue scale of 16.1±5.8 indicating a pale yellow color. Water-treated dentin that was stained with toluidine blue had a significantly (p<0.05) different chroma value of −27.4±3.0 indicating a color shift to blue. Dentin that was cationic polymer treated prior to dye staining had a chroma reading of 8.4±7.7, this was significantly (p<0.05) different from both the chroma value for unstained dentin and the value for dentin that was stained without cationic polymer treatment indicating a small but detectable color shift from yellow to blue.

### Example 3

### Cationic Beads

SEM observations of polished, EDTA-etched dentin washed with water and then treated with a dispersion of 0.52 μm diameter cationic beads revealed a surface heavily covered with beads. Beads could be seen covering the intertubular dentin, the orifices of some of the dentinal tubules and adhering to the tubule walls below the surface. In contrast, dentin in the half of the slice treated with the cationic polymer prior to the application of the beads showed few beads on the dentin surface or in the tubules. The heavy coating of the dentin with beads on the half of the slice exposed to water prior to the cationic beads and the lack of bead attachment in slice halves treated with cationic polymer solution was seen in the four other slices examined in the SEM. Particle counts obtained from water-treated and polymer-treated dentin surfaces that were then exposed to the cationic beads indicated that polymer treatment significantly reduced the number of beads adhering to the dentin surface. Quantitatively, 649±334.1 cationic beads per high power surface adhered to water treated dentin as opposed to only 9.8±4.3 beads per high power field in the disk halves that were treated with the cationic polymer prior to bead application. This difference was statistically significant (p<0.05). Examination of the polymer-treated dentin by SEM, revealed an appearance typical of etched dentin, as the cationic polymer coating could not be observed under the SEM.

### Example 4

### Anionic Beads

Polished, EDTA-etched dentin treated first with water, then with a dispersion of 0.45 μm diameter anionic beads were observed under the SEM to be uniformly coated with beads. While few beads were observed over the tubule orifices, beads were observed to line the walls of some of the tubules. In an additional five specimens exposed to water prior to treatment with the anionic beads, the same pattern of bead attachment was observed; a uniform coating of the dentin surface with beads. Treatment of the other half of the same dentin slice with the cationic polymer prior to application of the anionic beads changed the pattern of bead attachment. Irregular clumps of beads interspersed with relatively bare areas were seen to cover the dentin surface. Some of the dentin surfaces treated with the cationic polymer were observed to be relatively free of beads. Other specimens had areas of high bead density interspersed with relatively bear areas. Overall cationic polymer treatment significantly (p<0.05) reduced the number of anionic beads seen to adhere to the dentin surface with 1012±163.63 anionic beads per high power field adhering to water-treated dentin verse 411±323.36 beads adhering to cationic polymer-treated dentin. The number of anionic beads adhering to non-polymer-treated dentin surfaces did not differ significantly from the number of cationic beads adhering to untreated dentin surfaces (p>0.05).

### Example 5

### Silica Particle Adhesion to Dentin and Coverage of Tubule Orifices

To further examine the use of porous or hollow beads in a dentinal drug delivery system, uniform 0.5 μm silica particles were examined using SEM. Dentin disks were polished and etched using standard methods. Three types of silica particles were examined: amino (—NH₂) beads having a weak positive charge at physiological pH; hydroxyl (—OH) beads, which would have a neutral charge; and acid (—COOH) beads that are strongly negatively charged. Two and five percent dispersions of the beads were prepared by high power sonication. The dispersions were applied to moist dentin surfaces with a foam brush for 1 minute. The specimens were then exposed to deionized water, allowed to dry and prepared for SEM observation. Two thousand power images were prepared for analysis. Three dentin specimens were treated with each suspension. In this study, the image analysis software, Image J, was used to examine tubule coverage by the bead dispersions.

Dentinal tubules were evident in this analysis. For dentin surfaces treated with 2% acid (—COOH) beads, the surface was covered with particles; however the tubules were relatively open. Previous investigations observed a similar pattern of dentin coverage with —COOH functional latex beads. For dentin surfaces treated with 2% —NH₂ beads, in addition to dense coverage of the surface the dentin, the tubules were covered. Similar to the amine functional beads, dentin treated with —OH bearing silica provided a dense coverage of the surface as well as beads occupying the orifices of the dentinal tubules. Treatment of the dentin with 5% suspensions of the beads yielded similar patterns of dentin and tubule coverage.

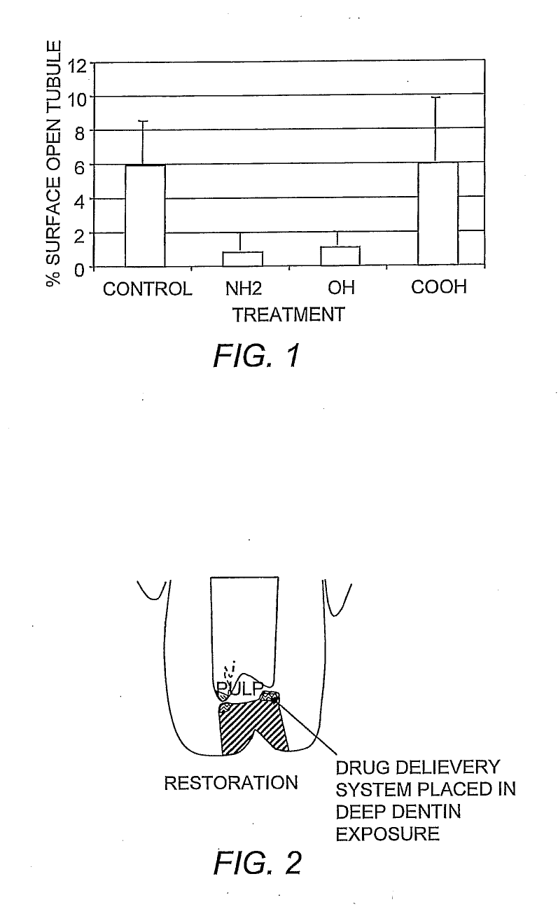

Using Image J, the open tubule percent of each dentin surface was calculated. Since images had to be converted into a binary format the tubule size may have been underestimated. FIG. 1 shows the percent open tubule (6 observations each group) for control (untreated), COOH—, NH₂— and OH-treated dentin. COOH bead treated and untreated dentin surfaces had the same proportion of the surface as open tubule. In contrast NH₂ and OH bead treated dentin had very small amounts of their surface as open tubule.

These results indicate that a variety of hydrophilic particles attach to dentin and resist water washing. Although the —COOH beads attached to the dentin surface, these beads were incapable of bridging over the tubule orifice in the same fashion as the other two bead types. Since the absolute magnitude of the surface charge is higher for —COOH than for NH₂, electrostatic repulsion may hinder the formation of these bridging structures.

These results indicate that positive or neutral beads will be of use as drug carriers to deliver drugs to dentin.

### Example 6

### Screen for Anti-Inflammatory Agents

The direct effect of drugs and lipids on inflammatory mediator production in RAW cells was determined. This analysis indicated that, compared to oleic acid, both aspirin and ibuprofen were effective inhibitors of LPS-evoked PGE2 production. Notably, the basal levels of PGE2 production were low in the absence of LPS stimulation (less than 1000 pg/ml). Similarly, aspirin effectively inhibited LPS-evoked NO production. DHA also inhibited NO production but to a lesser extent than aspirin. Measurable amounts of NO were produced in the absence of LPS stimulation (˜15 μM). Both aspirin and DHA appeared to cause a small (not statistically significant) reduction in this spontaneous NO production. This analysis indicated that 5 mM aspirin significantly reduced NO production, whereas 0.5 mM and lower concentrations (0.05, 0.005, and 0.0005 mM) did not significantly reduce LPS-evoked NO production. Although both aspirin and DHA treatment reduced NO production, combined treatment produced no evidence of a synergistic effect. In contrast to aspirin, ibuprofen had no effect on LPS-evoked NO production, even at a concentration of 50 μM, which inhibited LPS-evoked PGE2 production. Levels of aspirin used in this study appeared to be non-toxic to cells.

Transdentinal diffusion of aspirin and ibuprofen was elucidated using the screening method of this invention. Diffusion experiments were performed with aspirin or ibuprofen solutions in the first chamber (the donor compartment). Following 24 hours of diffusion, fluid was withdrawn from the second chamber and applied to RAW cells. When these RAW cells where stimulated with LPS, PGE2 production was significantly reduced by the diffusate of both the aspirin and ibuprofen experiments. In aspirin diffusion experiments LPS-evoked NO production was also depressed, whereas ibuprofen diffusion experiments did not attenuate LPS-stimulated NO production.

These experiments examined the ability of drugs to suppress inflammatory pathways evoked by bacterial endotoxin challenge. LPS injection induces severe local and systemic inflammatory responses. Aspirin demonstrated unexpected utility as it inhibited both PGE2 and NO production and diffused through dentin in sufficient quantities to suppress these important inflammatory pathways. Like aspirin, ibuprofen also diffused though dentin and inhibited PGE2 production. However, ibuprofen did suppress NO production. DHA, a ω-3 fatty acid, also inhibited LPS-provoked NO production but to a lesser extent than aspirin. This property was not shared by oleic acid a monounsaturated fatty acid. These results highlight the utility of ω-3 fatty acids in modulating the inflammatory process without suppressing it to the same extent as NSAID agents.

What is claimed is:

1. A dentinal drug delivery composition comprising cationic and/or neutral porous particles containing an effective amount of a therapeutic agent, said particles in admixture with a carrier suitable for attachment of the particles to the dentin.

2. The composition of claim 1, wherein the porous particle comprises silicon or latex.

3. The composition of claim 1, wherein the therapeutic agent is an anti-inflammatory drug that controls pulpal inflammation.

4. The composition of claim 3, wherein the anti-inflammatory drug is 2-amino-3-benzoylbenzeneacetamide, acetylsalicyclic acid, ibuprofen, or an ω-3 fatty acid.

5. The composition of claim 1, wherein the therapeutic agent is an antibiotic.

6. The composition of claim 1, wherein the therapeutic agent is an analgesic.

7. A method for providing dental treatment comprising administering to a subject in need thereof, the dentinal drug delivery composition of claim 1, thereby providing a dental treatment to the subject.

8. A method for identifying an anti-inflammatory agent for decreasing inflammation in dental pulp comprising
(a) contacting a coronal surface of an isolated dentin sample with a test agent,
(b) incubating the test agent and dentin sample so that diffusate is formed,
(c) contacting an isolated dental pulp cell with the diffusate, and
(d) determining whether the diffusate comprises an amount of test agent capable of decreasing the expression or activity of at least one inflammatory mediator of the dental pulp cell thereby identifying an anti-inflammatory agent for decreasing inflammation in dental pulp.

9. The method of claim 8, wherein the test agent is a non-steroidal anti-inflammatory drug.

10. The method of claim 8, wherein the test agent is an ω-3 fatty acid.

11. An anti-inflammatory agent identified by the method of claim 8.
